(19) United States
(12) Patent Application Publication
(10) Pub. No.: US 2008/0221344 A1
(43) Pub. Date: Sep. 11, 2008

(54) Biodiesel fuel and method of manufacture therefor

(76) Inventors: Rodney J. Gleason, Highland, MI (US); Albert S. Worrell, Milford, MI (US)

Correspondence Address: The Weintraub Group, P.L.C., Suite 240, 32000 Northwestern Highway, Farmington Hills, MI, 48334, US

(21) Appl. No.: 12/074,441

(22) Filed: Mar. 4, 2008

Related U.S. Application Data
(60) Provisional application No. 60/904,946, filed on Mar. 5, 2007.

(51) Int. Cl. C07C 51/00

(52) U.S. Cl. 554/174

(57) ABSTRACT

A method for producing biofuel by a transesterification reaction of an alcohol and a triglyceride such as an oil or fat is carried out at supercritical conditions in a reactor using a stoichiometric excess of alcohol. The reaction products of biofuel and gaseous mixture of glycerine and alcohol are re-cycled through a series of pre-heaters to sequentially raise the temperature and pressure of the reaction mixture prior to delivery to the reactor. Any excess alcohol after separating and recovering gaseous glycerin therefrom is recycled and mixed with “fresh” alcohol. Preferably, the process is a non-catalytic continuous process.

### CROSS-REFERENCE TO RELATED APPLICATIONS

This application is a completion application of co-pending U.S. Provisional Application Ser. No. 60/904,946, filed Mar. 5, 2007, for “Biodiesel Fuel and Method of Manufacture,” the entire disclosure of which is hereby incorporated by reference.

### BACKGROUND OF THE INVENTION

1. Field of the Invention

The present invention pertains to biodiesel fuels. More particularly, this invention relates to the manufacture of biodiesel fuels. Even more particularly, the present invention pertains to a transesterification process for the manufacture of biodiesel fuels and methods of recovery therefor.

2. Description of the Prior Art

As is known to those skilled in the art to which the present invention pertains the growth in the utilization of biodiesel fuels has proliferated. With the demand for reduction in petroleum oil consumption there is a growing recommendation for turning to renewable fuel sources. In this regard, the art has developed means and methods for the manufacture of biofuels and, in particular, biodiesel fuels.

Generally, such fuels are prepared by the transesterification of a triglyceride(s) either: (a) in the presence of a catalyst or (b) by using the alcohol in a supercritical condition. In a transesterification reaction, usually, a fat or oil from any suitable source, such as corn oil or the like is reacted with the alcohol to form a fatty acid ester which can then be successfully deployed as a fuel. This transesterification process is well-known such as described in U.S. Pat. Nos. 6,570,030; 6,187,939; 6,090,959, and U.S. Publication No. 2006/0025620, the disclosures of which are hereby incorporated by reference.

The efficiency of such processes is not satisfactory. In the catalytic reaction, because of the presence of the catalyst and the potential for soap by-products, the efficiency of such a reaction system is somewhat denigrated. Similarly, in the non-catalytic reaction, because excess stoichiometric quantities of alcohol are used, the economics of the process lessen the practicality of using same.

Thus, there exists a need for improvements in the manufacturing process whereby the excess reactants can be recycled and reused. It is to this to which the present invention is directed.

### SUMMARY OF THE INVENTION

In accordance with the present invention, a biofuel is prepared by the transesterification of a triglyceride, i.e. fat or oil with an alcohol, the alcohol being in a supercritical state.

According to the present invention, and in a preferred embodiment, a stoichiometric excess of an alcohol is reacted with a vegetable oil in a suitable reactor the reactor being maintained at supercritical conditions, i.e. at a temperature above about 180° and a pressure greater than about 1450 psi. Prior to delivering the reactants to the reactor they are passed through a series of pre-heaters. The pre-heaters use recycled reaction products to pre-heat the reactant.

According to the present invention, after leaving the reactor, the resulting reactor products are passed into a first flash drum after first passing through a proximal pre-heating at conditions that condense the biodiesel fuel while leaving the excess alcohol and glycerol by-product in a gaseous state. Liquid fuel is collected from the bottom of the drum and recycled to a first intermediate pre-heater from where it is collected, while the alcohol and glycerol are transferred to a second flash drum.

The second flash drum is set to a temperature and pressure that cause the glycerol to condense, while leaving the alcohol in a gaseous state. The glycerol is collected from the drum and the gaseous alcohol is then recycled, condensed and mixed with fresh alcohol which, in turn, is then admixed with the selected oil or fat and, then, pumped into the reactor through the pre-heaters.

Preferably, the reaction is a continuous process, although a batch process could be employed.

It should be noted that although the catalytic reaction can be used, it is preferred that the supercritical reaction be employed for efficiency and not having to recover the catalyst.

For a more complete understanding of the present invention, reference is made to the following detailed description and accompanying drawing. In the drawing, like reference characters refer to like parts throughout the several view in which:

### BRIEF DESCRIPTION OF THE DRAWINGS

FIG. 1 is a flow chart showing a first embodiment of the present process, and

FIG. 2 is a flow chart showing a second embodiment hereof.

### DETAILED DESCRIPTION OF THE PREFERRED EMBODIMENT

As noted above, the present invention contemplates a continuous process wherein an alcohol and a triglyceride, i.e. an oil or a fat are reacted together in a suitable reactor or furnace, wherein the furnace maintains the alcohol at a supercritical temperature, i.e. above 180° C. and at a pressure greater than about 1450 psi.

More particularly, and in a first embodiment hereof, the present invention contemplates the mixing together of an alcohol source and a triglyceride source wherein the admixture is then pumped, under pressure, into a reactor maintained at elevated temperature and pressure.

Prior to entering the reactor, the reactants are fed through a series of pre-heaters. The pre-heaters use the heat of the reactants to pre-heat the reactor by recycling the reactants. Thus, after the reaction is complete, the reactants are then inputted or transferred into a proximal pre-heater and, therefrom, into a first flash drum.

The biodiesel fuel is condensed in the first drum while gaseous alcohol and glycerol by-product remain in the gaseous state. These gaseous components are, then, transferred into a second flash drum where the glycerol is condensed, drawn off and collected while the alcohol remains in a gaseous state. The gaseous alcohol is then passed through at least one medial or intermediate pre-heater to liquefy the alcohol and then, recycled, to an alcohol feed tank and mixed with fresh alcohol.

The biofuel from the first flash drum is transferred to second intermediate pre-heater where it is used to pre-heat the reaction mixture. It is then drawn off and collected.

In carrying out the reaction, the first component as noted, is an alcohol or alkanol. Typically, these alcohols correspond to the formula:

CₙH₂ₙ₋₁(OH)

wherein n ranges from about one to about ten and, preferably, from about one to about four.

Among the useful alcohols are methanol, ethanol, n-propanol, isopropanol, n-butanol, isobutanol and the like, as well as mixtures thereof.

Preferred alcohols are methanol, ethanol, n-propanol, isopropanol and the like and mixtures thereof. Most preferred is methanol, alone, or in admixture with any of the other alkanols.

Where an alcohol admixture is used, the admixture will comprise a methanol to other alkanol volumetric ratio of about 1:00 to about 99:1, and preferably, at least a 50:50 volumetric ratio of methanol to other alkanol.

The second component in the present process comprises a triglyceride(s), i.e. a fat or oil. Useful and preferred are vegetable oils derived from renewable sources such as corn, soybean, rape seed or the like. However, other vegetable oils can be used such as sunflower seed oil, peanut oil and the like. Preferably, because of its abundance, soybean oil is used.

Useful fats include animal fat.

In carrying out the reaction, a stoichiometric excess of alcohol is used to ensure maximum transesterification, i.e. about 95%.

By carrying out the reaction in a supercritical state, usually at a temperature greater than 180° C. and at a pressure greater than about 1450 psi, the need for a catalyst is eliminated.

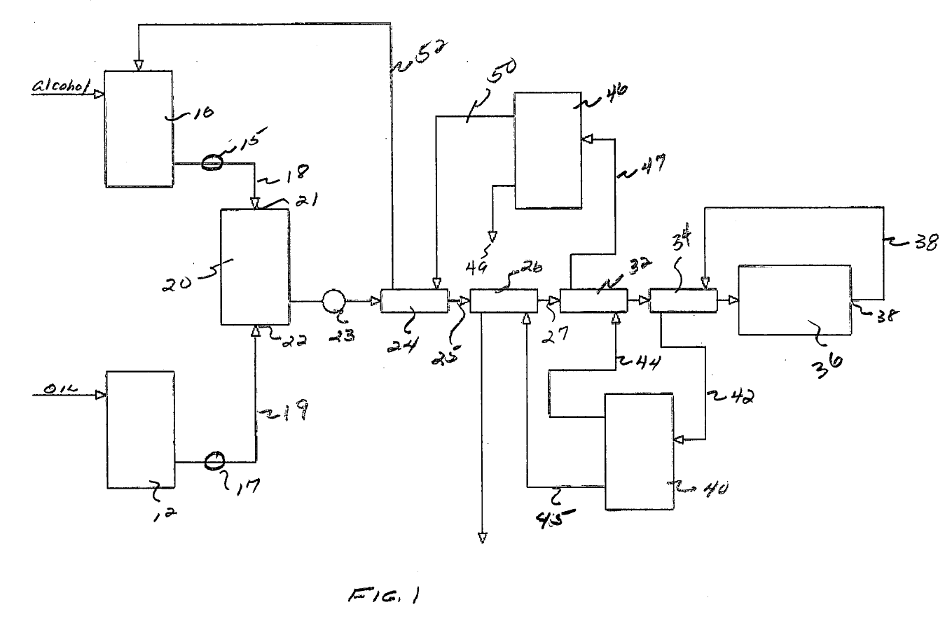

Referring now to the drawing, and, in particular FIG. 1, there is shown a flow chart for carrying out a first embodiment of the present process. As shown, initially, there is provided an alcohol source and a triglyceride source depicted as “oil” source comprising feed tanks 10 and 12, respectively.

The alcohol and the oil are each pumped under pressure, via pumps 15 and 17 ultimately into a mixing chamber 20 through delivery lines 18 and 19. The lines 18 and 19 are in fluid communication with the chamber 20 via inlets 21 and 22. The mixing chamber agitates the reactants to form a homogenous mixture maintained under a pressure of less than 1450 psi.

After mixing, the oil and alcohol mixture is delivered to a first or distal pre-heater 24 under pressure via a pump 23.

The pre-heater elevates the reactants to a temperature below the supercritical temperature and a pressure below 1450 psi.

As shown, the pre-heater 24 is in fluid communication with a first intermediate pre-heater 26 via delivery line 25. Delivery line 25 feeds the first intermediate pre-heater 26 through the pump 23.

In the intermediate pre-heater 26, the reaction mixture is further heated to a temperature, but still below about 180° C., while maintaining an elevated pressure, but below 1450 psi. As explained subsequently hereinbelow, each of the pre-heaters uses a countercurrent flow to transfer heat through a recycling of reaction products.

From the first intermediate pre-heater, the reaction mixture is, preferably, transferred to a second intermediate pre-heater 32 via delivery line or tube 27 where the temperature of the reaction mixture is elevated still higher but below the lash temperature of the alcohol while maintaining a pressure of below 1450 psi.

As shown in FIG. 1, the still further heated reaction mixture is then transferred or delivered to a proximal pre-heater 34 where the reactants are further heated while still elevating the pressure.

From the proximal pre-heater 34, the reaction mixture enters the reactor or furnace 36. As noted, the reactor 36 is a continuous reactor maintained at supercritical conditions, i.e. at a high pressure, a pressure greater than about 1450 psi and at a temperature greater than 180° C. and, preferably greater than 300° C. The reactor is equipped with means for stirring, such as a static mixer (not shown), to ensure that the reactants are maintained in turbulent mixing to ensure contact therebetween.

After the reaction is complete, usually, in about 5 to about 10 minutes, depending on the flow rates into the reactor, the resulting products i.e. (a) biofuel and (b) a glycerol-alcohol gaseous mixture, flow, while being maintained under pressure, exit from a reactor outlet 38 into the proximal pre-heater 34 via delivery line 38. Heat from the reaction product is transferred to the reaction mixture. From the proximal pre-heater, the reaction products flow into a first flash drum 40 via delivery line 42 while the heated reactants in the pre-heater 34 enter the reactor or furnace 36.

Flash drums are well known and commercially available and, generally, comprise an elongated tubular member having separation plates contained therewithin to permit liquid condensates to be collected at the bottom of the drum with the gaseous components rising to the top.

Generally, in the practice of the present invention the first flash drum 40 is maintained at a temperature less than that of the reactor, i.e. from about 280° C. to about 290° C. and at a pressure less than that of the reactor pressure but greater than atmospheric. In this manner, the hot liquid alkyl fatty acid ester biofuel condenses and is collected at the bottom of the drum and withdrawn therefrom and recycled into the first intermediate pre-heater 26 via delivery line 45 to transfer its heat to the reactants.

The excess alcohol and glycerol remain in a gaseous state and are transported through a delivery line 44 to the second intermediate pre-heater 32 via conduit 44 where some of its heat is transferred to the reactants. After the heat transfer, the glycerol-alcohol mixture is still gaseous.

From the second intermediate pre-heater 32, the alcohol-glycerol mixture is delivered to a second flash drum 46 via delivery line 47. The drum 46 is similar to the flash drum 40. The second flash drum 46 is maintained at a temperature ranging from about 66° C. to about 289° C. and at a pressure less than that of the first flash drum 26 but greater than atmospheric. In the second drum 46, glycerol is condensed and collected at the bottom as at 49 while gaseous alcohol is further recycled. As shown, this alcohol is, then, recycled via delivery line 50 to the distal pre-heater 24, where it loses sufficient heat to be a liquid. The so recovered liquid alcohol is, then, re-cycled to the feed tank 10 via delivery line 52, and admixed with fresh alcohol thereat.

According to the present method, the recovery of and reuse of the stoichiometric excess of alcohol lowers the amount of “fresh” alcohol necessary to be used in the reaction, while effectively recovering the biofuel.

Glycerol, which is recovered as a by-product, can then be used for various other compositions in which glycerol or glycerine is a component.

Furthermore, by using the heat of the reaction products to pre-heat the reactants or much more efficient process is achieved.

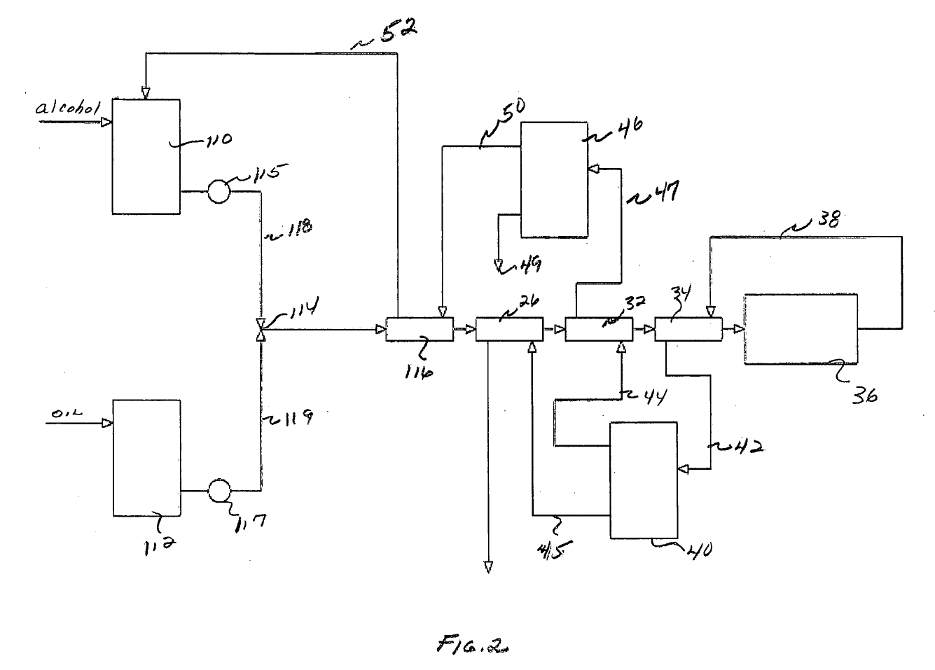

Referring now to FIG. 2, there is depicted therein a second embodiment hereof. According to this embodiment, each of the feed tanks 110 and 112 feed directly to a metering pump 114 via pumps 115, 117 which, in turn, pumps the appropriately rationed oil-alcohol reaction mixture to the distal pre-heater 116. In all other respects the process sis the same as described with respect to the first embodiment.

Alternatively, and preferably, and in a second embodiment hereof, the alcohol and the triglyceride feedstock are each, individually, pumped under pressure into the reactor through a common staging area. In either event, the reactants flow through the system, preferably, in a turbulent state.

It should be noted that each of the pre-heaters comprise a counter-current or counterflow heat exchanger. Such are well known and commercially available and, generally, include concentric tubes formed from a suitable non-reactive material having sufficient thermal conductively to enable sufficient heat transfer from the reaction product(s) to the reactants. Any such heat exchanger can be used herein so long as it withstands the processing parameters and is non-reactive

From the preceding, it is apparent that there has been described herein an improved process for the production of biodiesel fuel.

It is contemplated that the present method will enable a 95% conversion rate. It should be noted that typically the alcohol and oil will be used in about a 2:1 feed rate into the reactor to provide the requisite excess alcohol.

As noted above, a batch process, with the same process parameters can be used with the amounts of reactants adjusted to accommodate the necessary stoichiometric excess of alcohol, while still recovering and recycling same.

Having thus described the invention what is claimed is:

1. A method for manufacturing a biofuel by a transesterification process comprising:
(a) providing an alcohol source;
(b) providing a triglyceride source;
(c) mixing together a triglyceride and an alcohol to form a reaction mixture;
(d) transferring the reaction mixture to at least a distal pre-heater to heat the mixture to a temperature below about 180° C. and at a pressure of below about 1450 psi;
(e) transferring the pre-heated reaction mixture to a reactor maintained at a temperature of greater than about 180° C. and at a pressure greater than 1450 psi to form:
(1) a liquid biofuel and
(2) a glycerine and excess alcohol gaseous mixture;
(f) collecting the so-produced biofuel;
(g) collecting the glycerine, and;
(h) recycling the excess alcohol to the alcohol source.

2. The method of claim 1 wherein a stoichiometric excess of alcohol is used.

3. The method of claim 2 which further comprises:
providing a proximal pre-heater in fluid communication with the distal pre-heater and the reactor, and a first flash reactor, transferring the reaction mixture from the first pre-heater to the proximal pre-heater and then to the reactor; and
after the reaction is complete, recycling the reaction products to the proximal pre-heater and therefrom to a first flash reactor drum.

4. The method of claim 3 wherein the proximal pre-heater is interposed the first pre-heater and the reactor, the second pre-heater being at a temperature and pressure greater than that of the first pre-heater but below about 180° C. and 1450 psi.

5. The method of claim 3 which further comprises:
providing a first intermediate pre-heater and a second intermediate pre-heater, the first intermediate pre-heater being in fluid communication with the distal pre-heater, and the second intermediate pre-heater being in fluid communication with the first intermediate pre-heater and the proximal pre-heater;
transferring the biofuel from the first flash drum to the first intermediate pre-heater to heat the reaction mixture to a temperature and pressure above the distal pre-heater but below that of the second intermediate pre-heater, transferring the alcohol and glycerine gaseous mixture from the first flash drum to the second intermediate pre-heater to pre-heat the reaction mixture to a temperature and pressure above that of the first intermediate pre-heater but below that of the proximal pre-heater, and collecting the biofuel from the first intermediate pre-heater.

6. The method of claim 5 which further comprises:
providing a second flash drum;
transferring the glycerin and alcohol mixture from the second intermediate pre-heater to the second flash drum to condense the glycerin; collecting the glycerin;
transferring the remaining gaseous alcohol to the distal pre-heater to pre-heat the reaction mixture;
delivering the so-recycled alcohol from the distal pre-heater to the source of alcohol; and
mixing the recycled alcohol with the alcohol source.

7. The method of claim 5 wherein:
transesterification the reactants are an alcohol and an oil, the alcohol corresponding to the formula:
CₙH₂ₙ₋₁(OH)
wherein n ranges from about 1 to about 10 and the oil is a vegetable oil.

8. The method of claim 7 wherein the method is a continuous process.

9. The method of claim 8 wherein:
each of the pre-heaters is a counter-current flow heat exchanger.

10. The method of claim 1 where the alcohol is methanol.

11. In a method for manufacturing a biofuel by a transesterification reaction of a triglyceride and a stoichiometric excess of an alcohol where the reaction is carried but at a supercritical state, the improvement which comprises:
recycling the reaction product through at least one heat exchanger to pre-heat the reactants; and
mixing the excess re-cycled alcohol with additional reactant alcohol.
